(19) United States
(12) Patent Application Publication
(10) Pub. No.: US 2022/0096418 A1
(43) Pub. Date: Mar. 31, 2022

(54) KETONE BODY PRODUCTION PROMOTING COMPOSITION

(71) Applicant: MEIJI CO., LTD., Tokyo (JP)

(72) Inventors: Kentaro NAKAMURA, Hachioji-shi, Tokyo (JP); Kinya ASHIDA, Hachioji-shi, Tokyo (JP); Akina SASAYAMA, Hachioji-shi, Tokyo (JP)

(73) Assignee: MEIJI CO., LTD., Tokyo (JP)

(21) Appl. No.: 17/426,319

(22) PCT Filed: Jan. 30, 2020
(86) PCT No.: PCT/JP2020/003386

§ 371 Date: Jul. 28, 2021

(30) Foreign Application Priority Data
Jan. 30, 2019 (JP) 2019-014199

(51) Int. Cl. A61K 31/22; A61P 3/00

(52) U.S. Cl. CPC A61K 31/22; A61P 3/00

(57) ABSTRACT

The present invention provides a ketone body production promoting composition comprising: as an active ingredient, a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms.

### TECHNICAL FIELD

The present invention relates to a ketone body production promoting composition.

### BACKGROUND ART

Ketone bodies are a general term for acetoacetic acid, β-hydroxybutyric acid, and acetone, and are produced from fatty acids in the liver in vivo. The brain can use ketone bodies in addition to dextrose (glucose) as an energy source, and the energy required by the brain (ATP) is usually produced by the metabolism of dextrose through the TCA cycle, whereas in the liver, fatty acids are decomposed into acetyl-CoA by β-oxidation, and then most of them are completely oxidized in the TCA cycle. On the other hand, when dextrose supply becomes insufficient due to abnormal sugar metabolism (diabetes), insufficient sugar intake (starvation), or the like, the liver produces acetoacetic acid from the above acetyl-CoA to provide an energy source for the brain, and part of the acetoacetic acid is converted to acetone by decarboxylation, and other part thereof is enzymatically reduced to β-hydroxybutyric acid, respectively. While acetone is volatile and easily excreted in the exhaled breath, the other ketone bodies produced (acetoacetic acid and β-hydroxybutyric acid) are transported to other organs including the brain (such as the heart, muscles, and the kidneys), returned to the acetyl-CoA in each cell, and metabolized through the TCA cycle to produce energy. Therefore, ketone bodies are important as an alternative energy source for dextrose, especially in the brain.

As described above, the blood ketone body concentration in vivo increases mainly due to the increase in ketone body production in the liver, and a low-carbohydrate, high-fat diet (so-called ketogenic diet) has long been known as a diet devised to produce a large amount of ketone bodies. Such a ketogenic diet is known to be useful for the treatment of intractable epilepsy and GLUT1 deficiency, the suppression of seizures in intractable epilepsy, and the like (NPL 1), and further has been reported in recent years to be effective in treating diabetes and obesity (weight loss), cancer, Alzheimer's disease, and the like (NPL 2).

As fatty acids that are the main raw materials for the ketone body production, most of the fatty acids that can be ingested as natural fatty acid oils and/or fats are fatty acids having 8 carbon atoms (caprylic acid), fatty acids having 10 carbon atoms (capric acid), and fatty acids having 12 carbon atoms (lauric acid). Among these, caprylic acid and capric acid are considered to be able to produce ketone bodies more efficiently than fatty acids having more carbon atoms due to differences in absorption and metabolic pathways. For example, it has been reported that administration of a glyceride of caprylic acid or capric acid increases the blood ketone body concentration in humans and non-human animals as compared with administration of a fatty acid having 14 or more carbon atoms (NPL 3). However, since oral administration of a glyceride of caprylic acid or capric acid is known to cause gastrointestinal symptoms such as diarrhea and vomiting, from the viewpoint of maintaining and improving QOL, a method is desired of increasing the blood ketone body concentration by a method other than simply administering the fatty acids.

Note that for the ketone body, for example, a method of direct oral ingestion, such as a nutritional supplement composition containing a basic amino acid salt of 3-hydroxybutyric acid described in Japanese Unexamined Patent Application Publication No. 2017-201906 (PTL 1), is also known, but tends to have difficulty in increasing the blood ketone body concentration as compared with the case of production in a living body as described above.

### CITATION LIST

### Patent Literature

[PTL 1] Japanese Unexamined Patent Application Publication No. 2017-201906

### Non Patent Literature

[NPL 1] “From the Basics to Practice of Ketogenic Diet,” edited by Tatsuya Fujii, SHINDAN TO CHIRYO SHA, Inc., 2011

[NPL 2] Maciej Gasior et al., Behav Pharmacol., 2006, vol. 17, p. 431-439

[NPL 3] Pi-Sunyer F X et al., Diabetes., 1969, vol. 18, p. 96-100

### SUMMARY OF INVENTION

### Technical Problem

The present invention has been made in view of the above problems, and an object thereof is to provide a novel ketone body production promoting composition capable of increasing the blood ketone body concentration.

### Solution to Problem

The present inventors have found that by administering (preferably orally administering) to a subject a fatty acid glyceride containing a saturated fatty acid having 6 or less carbon atoms as a constituent fatty acid (C6 or less fatty acid glyceride), it is possible to significantly increase the blood ketone body concentration. In addition, among the ketone bodies that can be present in blood (acetoacetic acid and β-hydroxybutyric acid), only acetoacetic acid is used for energy production, and β-hydroxybutyric acid is used for energy production only after conversion to acetoacetic acid. Additionally, acetoacetic acid is expected to suppress weight gain, suppress body fat accumulation, decompose body fat, and the like. Therefore, acetoacetic acid has received particular attention. The present inventors have found that the above-mentioned administration can increase the blood acetoacetic acid concentration particularly significantly, and have completed the present invention.

Specifically, the present invention provides the following.
[1] A ketone body production promoting composition comprising: as an active ingredient, a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms.
[2] The ketone body production promoting composition according to [1], wherein the C6 or less fatty acid glyceride is a fatty acid triglyceride containing three molecules of fatty acids as constituent fatty acids.
[3] The ketone body production promoting composition according to [1] or [2], wherein the amount of the saturated fatty acid having 6 or less carbon atoms as a constituent fatty acid of the C6 or less fatty acid glyceride is 5% by mass or more when a total amount of the constituent fatty acids of the fatty acid glyceride contained in the ketone body production promoting composition is set to 100% by mass.
[4] The ketone body production promoting composition according to any one of [1] to [3], wherein the saturated fatty acid having 6 or less carbon atoms has 4 to 6 carbon atoms in the C6 or less fatty acid glyceride.
[5] The ketone body production promoting composition according to any one of [1] to [4], wherein the ketone body is acetoacetic acid, and the ketone body production promoting composition is a composition used for promoting production of acetoacetic acid.
[6] The ketone body production promoting composition according to any one of [1] to [5], which is a composition for increasing a blood ketone body concentration.
[7] The ketone body production promoting composition according to any one of [1] to [6], which is a composition for preventing or ameliorating a disease or symptom in which an increase in blood ketone body concentration is effective.
[8] The ketone body production promoting composition according to any one of [1] to [7], which is a composition selected from the group consisting of pharmaceutical compositions, quasi-drug compositions, food and drink compositions, and feed compositions.
[9] A method for increasing a blood ketone body concentration, comprising: administering the ketone body production promoting composition according to any one of [1] to [8] to a human or non-human animal.
[10] Use of a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms, or the ketone body production promoting composition according to any one of [1] to [8], for increasing a blood ketone body concentration.
[11] A method for preventing or ameliorating a disease or symptom in which an increase in blood ketone body concentration is effective, comprising: administering the ketone body production promoting composition according to any one of [1] to [8] to a human or non-human animal.
[12] Use of a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms, or the ketone body production promoting composition according to any one of [1] to [8], for preventing or ameliorating a disease or symptom in which an increase in blood ketone body concentration is effective.
[13] Use of a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms, for producing a ketone body production promoting composition.

### Advantageous Effects of Invention

According to the present invention, it is possible to provide a novel ketone body production promoting composition capable of increasing the blood ketone body concentration. In addition, according to the fatty acid glyceride (C6 or less fatty acid glyceride) containing a saturated fatty acid having 6 or less carbon atoms contained as an active ingredient in the ketone body production promoting composition of the present invention as a constituent fatty acid, gastrointestinal symptoms (such as diarrhea, vomiting, nausea, irritation, pain, fever, retching, bloating, and belch) are less likely to be caused by oral administration compared with fatty acid glycerides containing saturated fatty acids having 8 to 10 carbon atoms as constituent fatty acids, so that the gastrointestinal symptoms can be suppressed.

### BRIEF DESCRIPTION OF DRAWINGS

FIG. 1 shows a plasma acetoacetic acid concentration-post administration elapsed time curve obtained by administering the test compositions prepared in Examples 1 and 2, Reference Example 1, and Comparative Examples 1 to 4 to rats.

FIG. 2 shows a plasma β-hydroxybutyric acid concentration-post administration elapsed time curve obtained by administering the test compositions prepared in Examples 1 and 2, Reference Example 1, and Comparative Examples 1 to 4 to rats.

FIG. 3 shows a total plasma ketone body concentration-post administration elapsed time curve obtained by administering the test compositions prepared in Examples 1 and 2, Reference Example 1, and Comparative Examples 1 to 4 to rats.

### DESCRIPTION OF EMBODIMENTS

Hereinafter, the present invention is described in detail according to preferred embodiments thereof.

A ketone body production promoting composition of the present invention comprises: as an active ingredient, a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms.

In the present invention, “ketone body production promoting” means that the production of ketone bodies in vivo is promoted, and the promotion of the production of ketone bodies in vivo can be confirmed by an increase in the concentration of ketone bodies in the circulating blood or plasma (hereinafter simply referred to as “blood ketone body concentration” in some cases) after administration (preferably oral administration) of the composition to the subject, as compared with that before the administration. In addition, since acetone is volatile and easily excreted in exhaled breath, in the present invention, a ketone body in vivo (hereinafter simply referred to as “ketone body” in some cases) is a general term for acetoacetic acid and β-hydroxybutyric acid. The degree of ketone body production promoting can be evaluated by the blood concentration of each ketone body. Specifically, after administration (preferably oral administration) of the composition to the subject, at least one of blood acetoacetic acid concentration, blood β-hydroxybutyric acid concentration, and a total concentration thereof (blood ketone body concentration) is measured over time. Evaluation can be performed by the maximum value (Cmax) of the blood ketone body concentration within a certain time after administration (preferably within 8 hours, more preferably within 6 hours, and further preferably within 4 hours), and/or the area under the blood ketone body concentration-time curve (AUC), which is the integral value of the blood ketone body concentration (y-axis)-post administration elapsed time (x-axis) curve at a certain time after administration (preferably 0 to 8 hours, more preferably 0 to 6 hours, and further preferably 0 to 4 hours).

Note that in the present invention, “in vivo” refers to the body of a subject for administration of the ketone body production promoting composition of the present invention, preferably the body of a human or non-human animal, and more specifically refers to the body of a mammal (including primates such as humans, monkeys, gorillas, hamadryas baboons, and chimpanzees; domestic animals such as horses, cows, water buffalo, sheep, goats, pigs, camels, and deer; and pet animals such as dogs and cats).

Further, in the present invention, the “ketone body production promoting composition” means a composition used for promoting the production of ketone bodies in vivo and a composition used for increasing the blood ketone body concentration of an administration subject.

In the present invention, the “C6 or less fatty acid glyceride” means a fatty acid glyceride which contains one to three molecules of fatty acids as constituent fatty acids and is a fatty acid glyceride in which at least one molecule of the constituent fatty acids is a saturated fatty acid having 6 or less carbon atoms.

In the present invention, the “fatty acid glyceride” means a glycerin fatty acid ester in which at least one of the three hydroxy groups of glycerol and the carboxy group of the fatty acid are ester-bonded (that is, one to three molecules of fatty acids are ester-bonded to one molecule of glycerol), and the “constituent fatty acid” means a fatty acid ester-bonded to the glycerol, that is, a fatty acid from which a fatty acid residue in the fatty acid glyceride is derived, or a fatty acid released by hydrolysis of the fatty acid glyceride.

The “C6 or less fatty acid glyceride” according to the present invention is the above-mentioned fatty acid glyceride, that is, a “fatty acid glyceride which contains one to three molecules of fatty acids as constituent fatty acids,” and may be a fatty acid monoglyceride having one constituent fatty acid, a fatty acid diglyceride having two constituent fatty acids, or a fatty acid triglyceride having three constituent fatty acids, but is preferably a fatty acid triglyceride. Further, when, in the Fischer projection, the hydroxy group bonded to the carbon atom (position 2) of glycerol is placed on the left and the upper part is defined as position 1 and the lower part as position 3, the “C6 or less fatty acid glyceride” according to the present invention preferably has a three-dimensional configuration in which the constituent fatty acids are bound in the order of positions 1, 3, and 2, more preferably at positions 1 and 3, and further preferably at all positions.

As the “C6 or less fatty acid glyceride” according to the present invention, it is necessary that at least one molecule of the constituent fatty acids in one molecule of fatty acid glyceride is a “saturated fatty acid having or less carbon atoms.” This makes it possible to significantly increase the blood ketone body concentration, particularly the blood acetoacetic acid concentration, as compared with, for example, the fatty acid triglyceride in which all three molecules of the constituent fatty acids are saturated fatty acids having 10 or more carbon atoms.

The saturated fatty acid having 6 or less carbon atoms according to the present invention may be linear or branched, but is preferably linear (saturated linear fatty acid). In addition, the number of carbon atoms is particularly preferably 4 to 6. When the number of carbon atoms is less than the lower limit, the flavor of the composition tends to be impaired. Meanwhile, when the upper limit is exceeded, the gastrointestinal symptoms tend to be caused at the time of oral administration, and it tends to be difficult to sufficiently increase the blood ketone body concentration, particularly the blood acetoacetic acid concentration. More specific examples of such saturated fatty acid having 6 or less carbon atoms include formic acid, acetic acid, propionic acid, butyric acid (butanoic acid), valeric acid (pentanoic acid), and caproic acid (hexanoic acid), and when two or more molecules of the constituent fatty acids are contained, one of them may be used alone or in combination of two or more. Among these, from the viewpoints of linear saturated fatty acid and natural occurrence, the saturated fatty acid having 6 or less carbon atoms according to the present invention is preferably butyric acid and/or caproic acid, and more preferably butyric acid or caproic acid.

In the “C6 or less fatty acid glyceride” according to the present invention, when the constituent fatty acids include an additional fatty acid other than the saturated fatty acid having 6 or less carbon atoms, such an additional fatty acid is not particularly limited, and may be saturated or unsaturated, and may be linear or branched, but is preferably saturated (saturated fatty acid) or linear (linear fatty acid), and more preferably a linear saturated fatty acid. In addition, the number of carbon atoms of the additional fatty acid may be 7 or more, preferably 7 to 30, more preferably 8 to 24, and further preferably 8 to 22. When the number of carbon atoms exceeds the upper limit, the composition tends to be poorly digested or unsuitable for food. Specific examples of such additional fatty acids include heptylic acid (heptanoic acid), caprylic acid (octanoic acid), nonanoic acid, capric acid (decanoic acid), lauric acid (dodecanoic acid), myristic acid (tetradecanoic acid), palmitic acid (hexadecanoic acid), palmitoleic acid (9-hexadecenoic acid), stearic acid (octadecanoic acid), oleic acid (cis-9-octadecenoic acid), vaccenic acid (11-octadecenoic acid), linoleic acid (cis,cis-9,12-octadecadienoic acid), (9,12,15)-linolenic acid (9,12,15-octadecatrienoic acid), (6,9,12)-linolenic acid (6,9,12-octadecatrienoic acid), arachidonic acid (5,8,11-eicosatetraenoic acid), eicosapentaenoic acid (icosa-5,8,11,14,17-pentaenoic acid, EPA), mead acid (5,8,11-eicosatrienoic acid), docosahexaenoic acid (docosa-4,7,10,13,16,19-hexaenoic acid, DHA), behenic acid (docosanoic acid), erucic acid, lignoceric acid (tetracosanoic acid), and melissic acid (triacontanoic acid), and when constituting two molecules of the constituent fatty acids, one of them may be used alone or in combination of two. When the constituent fatty acids include the above-mentioned additional fatty acid, it is preferably at least one selected from the group consisting of caprylic acid, capric acid, lauric acid, and stearic acid.

In the “C6 or less fatty acid glyceride” according to the present invention, at least one molecule of the constituent fatty acids may be the above-mentioned saturated fatty acid having 6 or less carbon atoms, but it is preferable that all of the constituent fatty acids, more preferably, the constituent fatty acids are three molecules, and two or all (three molecules) of the molecules are each the above-mentioned saturated fatty acid having 6 or less carbon atoms, and it is further preferable that all of them are each the above-mentioned saturated fatty acid having 6 or less carbon atoms. In addition, when the saturated fatty acid having 6 or less carbon atoms constitutes two or more molecules of the constituent fatty acids, they may be different from each other, but it is more preferable that all (preferably 3 molecules) of them are the same saturated fatty acid having 6 or less carbon atoms. More specifically, butyric acid triglyceride and caproic acid triglyceride are preferable as such C6 or less fatty acid glycerides.

In addition, as the “C6 or less fatty acid glyceride” according to the present invention, the total number of carbon atoms of the constituent fatty acids in one molecule of the fatty acid glyceride is preferably 3 to 66, more preferably 9 to 50, and further preferably 12 to 36. When the total number of carbon atoms of the constituent fatty acids is less than the lower limit, the flavor of the composition tends to be impaired. Meanwhile, when the upper limit is exceeded, the composition tends to be poorly digested or unsuitable for food.

In addition, in the “C6 or less fatty acid glyceride” according to the present invention, as the amount of the saturated fatty acid having 6 or less carbon atoms, the amount of the saturated fatty acid having 6 or less carbon atoms as a constituent fatty acid of the C6 or less fatty acid glyceride contained in the ketone body production promoting composition of the present invention has a lower limit value of preferably 5% by mass, more preferably 10% by mass, and further preferably 15% by mass when the total amount of the constituent fatty acids of all fatty acid glycerides contained in the ketone body production promoting composition is set to 100% by mass (including the total amount of the constituent fatty acids of the C6 or less fatty acid glyceride contained in the composition as well as the amount of the constituent fatty acids of additional fatty acid glycerides when the composition also contains the following additional fatty acid glycerides other than the C6 or less fatty acid glyceride). The upper limit value here is preferably 60% by mass, more preferably 80% by mass, and further preferably 100% by mass. In addition, these lower limit values and upper limit values can be arbitrarily combined (for example, 5 to 60% by mass, 5 to 100% by mass, 10 to 80% by mass, 15 to 100% by mass, and 15 to 60% by mass). When the amount of the saturated fatty acid having 6 or less carbon atoms is less than the lower limit, it tends to be difficult to sufficiently increase the blood ketone body concentration, particularly the blood acetoacetic acid concentration. Note that in the present invention, the amount of the constituent fatty acids of the fatty acid glyceride contained in the composition (including the amount of the saturated fatty acid having 6 or less carbon atoms as a constituent fatty acid of the C6 or less fatty acid glyceride) means the mass obtained by converting the mass of the fatty acid residue of the fatty acid glyceride into the mass of the fatty acid from which it is derived, or the mass of the fatty acid released by hydrolysis of the fatty acid glyceride.

The “C6 or less fatty acid glyceride” according to the present invention may be an extract from a natural product or a chemically synthesized product. As the natural product, the “C6 or less fatty acid glyceride” according to the present invention exists as an oil and/or fat contained in dairy products such as milk, sheep milk, and goat milk; and an oil and/or fat collected from a plant such as a palm family plant such as coconut or palm fruit, and it is possible to use extracts, crude purified products, purified products, and the like, which are appropriately extracted from these by a known method or a method similar thereto and purified as necessary. The chemically synthesized product is not particularly limited, and can be appropriately synthesized by a known method or a method similar thereto. In addition, as the “C6 or less fatty acid glyceride” according to the present invention, a commercially available product can be appropriately used.

As the “C6 or less fatty acid glyceride” contained as an active ingredient in the ketone body production promoting composition of the present invention, one of the C6 or less fatty acid glycerides according to the present invention may be used alone or two or more thereof may be used in combination, but it contains preferably a fatty acid triglyceride in which at least one molecule of the constituent fatty acids is a saturated fatty acid having or less carbon atoms, more preferably a fatty acid triglyceride in which at least two molecules of the constituent fatty acids are each a saturated fatty acid having 6 or less carbon atoms, further preferably a fatty acid triglyceride in which all (three molecules) of the constituent fatty acids are each a saturated fatty acid having 6 or less carbon atoms, further preferably a fatty acid triglyceride in which all of the constituent fatty acids are each a saturated fatty acid having 4 to 6 carbon atoms, and particularly preferably a butyric acid triglyceride and/or a caproic acid triglyceride. In the ketone body production promoting composition of the present invention, the content of these preferable C6 or less fatty acid glycerides (in the case of a mixture of two or more kinds, the total content thereof) is preferably 5% by mass or more, more preferably 10% by mass or more, and further preferably 50 to 100% by mass, based on the total content of the C6 or less fatty acid glyceride according to the present invention.

The ketone body production promoting composition of the present invention may be composed only of the above-mentioned C6 or less fatty acid glyceride according to the present invention, or may contain the C6 or less fatty acid glyceride according to the present invention and additional components as long as the effects of the present invention are not impaired. In addition, when the above-mentioned C6 or less fatty acid glyceride according to the present invention is contained, the content thereof is not particularly limited. Although it cannot be said unconditionally because it is appropriately adjusted according to the form of the following compositions, the purpose, subject, method, dose, and the like for administering the composition, the content of the C6 or less fatty acid glyceride according to the present invention in the ketone body production promoting composition of the present invention (in the case of a mixture of two or more kinds, the total content thereof, the same applies hereinafter) is usually preferably 0.1% by mass or more, more preferably 5% by mass or more, and further preferably 10% by mass or more. More specifically, the upper limit value of the content is 100% by mass, preferably 90% by mass, and more preferably 80% by mass. In addition, these lower limit values and upper limit values can be arbitrarily combined (for example, 0.1 to 100% by mass, more preferably 5 to 90% by mass, and further preferably 10 to 80% by mass).

As an example, when the ketone body production promoting composition of the present invention is, for example, a dairy product as a food and drink composition for ketone body production promoting or blood ketone body concentration increasing (for example, one stipulated in Japan's “Ministerial Ordinance on Milk and Milk Products Concerning Compositional Standards, etc. (Milk etc. Ordinance)”), the content of the C6 or less fatty acid glyceride is preferably 0.1% by mass or more, more preferably 0.5% by mass or more, and further preferably 1% by mass or more. More specifically, the upper limit value of the content of the C6 or less fatty acid glyceride is 10% by mass, preferably 6% by mass, and more preferably 5% by mass. In addition, these lower limit values and upper limit values can be arbitrarily combined (for example, 0.1 to 10% by mass, more preferably 0.5 to 6% by mass, and further preferably 1 to 5% by mass).

In addition, as long as the ketone body production promoting composition of the present invention contains the above-mentioned C6 or less fatty acid glyceride according to the present invention, the additional components are not particularly limited, and can be appropriately adjusted according to the form of the composition, the purpose, subject, method, dose, and the like for administering the composition as long as the effects of the present invention are not impaired, but from the viewpoint of sufficiently increase the blood ketone body concentration except for the subject of abnormal sugar metabolism, it is preferable to use a low-carbohydrate high-fat composition, that is, a composition having substantially no or low content of carbohydrates other than dietary fibers (that is, sugars) and a high content of lipids.

Examples of the sugar include monosaccharides, disaccharides, oligosaccharides, and polysaccharides, examples of the monosaccharides include glucose (dextrose), fructose (fruit sugar), and galactose, examples of the disaccharides include maltose (malt sugar), sucrose (cane sugar), and lactose (milk sugar), examples of the oligosaccharides include galactooligosaccharides, fructooligosaccharides, and mannan oligosaccharides, and examples of the polysaccharides include starch (amylose, amylopectin) and glycogen, and one of these may be used alone or in mixture of two or more.

When the low-carbohydrate high-fat composition contains the sugar, the content thereof (in the case of a mixture of two or more kinds, the total content thereof, the same applies hereinafter) is preferably 15% by mass or less, more preferably 13% by mass or less, and further preferably 10% by mass or less. More specifically, the lower limit value of the content is 0% by mass, preferably 3% by mass, and more preferably 5% by mass. In addition, these lower limit values and upper limit values can be arbitrarily combined (for example, 0 to 15% by mass, preferably 3 to 13% by mass, and more preferably 5 to 10% by mass).

In addition, in this case, when the total calorie of the low-carbohydrate high-fat composition is set to 100 kcal, the lower limit value of the calorie content of the sugar (in the case of a mixture of two or more kinds, the total calories thereof, the same applies hereinafter) can be 0 kcal, preferably 2 kcal, and more preferably 4 kcal. In addition, the upper limit value of the calorie content can be 10 kcal, preferably 8 kcal, and more preferably 6 kcal, and these lower limit values and upper limit values can be arbitrarily combined (for example, 0 to 10 kcal, preferably 2 to 8 kcal, and more preferably 4 to 6 kcal).

The lipids contain the C6 or less fatty acid glyceride according to the present invention, and may contain a lipid other than the C6 or less fatty acid glyceride as long as the effects of the present invention are not impaired. Examples of the lipid other than the C6 or less fatty acid glyceride according to the present invention include additional fatty acid glycerides in which the constituent fatty acids are composed only of additional fatty acids other than the saturated fatty acid having 6 or less carbon atoms (for example, fatty acid glycerides in which all of the constituent fatty acids are saturated or unsaturated fatty acids having 7 or more carbon atoms (for example, fatty acids listed as additional fatty acids above)), phospholipids, and glycolipids, and one of these may be used alone or in combination of two or more.

The lower limit value of the lipid content in the low-carbohydrate high-fat composition (including the content of the C6 or less fatty acid glyceride according to the present invention, in the case of a mixture of two or more kinds, the total content thereof, the same applies hereinafter) can be 10% by mass, preferably 20% by mass, and more preferably 30% by mass. In addition, the upper limit value of the content can be 80% by mass, preferably 60% by mass, and more preferably 50% by mass, and these lower limit values and upper limit values can be arbitrarily combined (for example, 10 to 80% by mass, preferably 20 to 60% by mass, and more preferably 30 to 50% by mass).

In addition, when the total calorie of the low-carbohydrate high-fat composition is set to 100 kcal, the lower limit value of the calorie content of the lipid (including the calorie of the C6 or less fatty acid glyceride according to the present invention, in the case of a mixture of two or more kinds, the total calories thereof, the same applies hereinafter) can be 30 kcal, preferably 35 kcal, and more preferably 40 kcal. In addition, the upper limit value of the calorie content can be 90 kcal, preferably 70 kcal, and more preferably 55 kcal, and these lower limit values and upper limit values can be arbitrarily combined (for example, 30 to 90 kcal, preferably 35 to 70 kcal, and more preferably 40 to 55 kcal).

The low-carbohydrate high-fat composition may further contain a protein as long as the effects of the present invention are not impaired. The protein is not particularly limited, and examples thereof include corn gluten, wheat gluten, soy protein, wheat protein, milk protein, animal protein (including collagen) obtained from meat or fish meat, egg white, and egg yolk, and one of these may be used alone or in mixture of two or more. Among these, milk protein is preferable. As the milk protein, it is possible to use a protein component contained in milk obtained from mammals including primates such as humans, monkeys, gorillas, hamadryas baboons, and chimpanzees; domestic animals such as horses, cows, water buffalo, sheep, goats, pigs, camels, and deer; and pet animals such as dogs and cats, and it is preferably at least one selected from the group consisting of whey proteins (such as α-lactalbumin (α-La), β-lactoglobulin (β-Lg), immunoglobulin, and lactoferrin), casein, and salts thereof (such as sodium caseinate, potassium caseinate, calcium caseinate, and magnesium caseinate). In addition, as the whey proteins, it is possible to use undiluted solution of whey (such as sweet whey and acid whey) and a concentrate, dried product (such as whey powder), and frozen product thereof; desalted whey; whey protein concentrate (WPC) and whey protein isolate (WPI), and the like.

When the low-carbohydrate high-fat composition contains the protein, the lower limit of the content (in the case of a mixture of two or more kinds, the total content thereof, the same applies hereinafter) can be 0% by mass, preferably 5% by mass, and more preferably 10% by mass. In addition, the upper limit value of the content can be 30% by mass, preferably 25% by mass, and more preferably 20% by mass, and these lower limit values and upper limit values can be arbitrarily combined (for example, 0 to 30% by mass, preferably 5 to 25% by mass, and more preferably 10 to 20% by mass).

In addition, in this case, when the total calorie of the low-carbohydrate high-fat composition is set to 100 kcal, the lower limit value of the calorie content of the protein (in the case of a mixture of two or more kinds, the total calories thereof, the same applies hereinafter) can be 0 kcal, preferably 3 kcal, and more preferably 5 kcal. In addition, the upper limit value of the calorie content can be 15 kcal, preferably 12 kcal, and more preferably 10 kcal, and these lower limit values and upper limit values can be arbitrarily combined (for example, 0 to 15 kcal, preferably 3 to 12 kcal, and more preferably 5 to 10 kcal).

When containing the above-mentioned C6 or less fatty acid glyceride according to the present invention as an active ingredient, the ketone body production promoting composition of the present invention can be used to promote the production of ketone bodies or to increase the blood ketone body concentration. In addition, since the ketone body production promoting composition of the present invention can particularly increase the blood acetoacetic acid concentration, it is more preferable to use it for promoting the production of acetoacetic acid or for increasing the blood acetoacetic acid concentration.

In addition, the ketone body production promoting composition of the present invention can be used to prevent or ameliorate a disease or symptom in which an increase in blood ketone body concentration is effective, for example, pediatric epilepsy, refractory epilepsy, glucose transporter 1 (GLUT1) deficiency, pyruvate dehydrogenase complex deficiency, neurodegenerative diseases (such as Alzheimer's disease and muscular dystrophy), mild cognitive impairment, Parkinson's disease, traumatic brain injury, cancer, depression, autism, migraine, amyotrophic lateral sclerosis, sleep attacks, diabetes, heart failure, myocardial infarction, angina pectoris, and obesity (weight gain or body fat accumulation), preferably pediatric epilepsy, refractory epilepsy, glucose transporter 1 (GLUT1) deficiency, pyruvate dehydrogenase complex deficiency, neurodegenerative diseases, mild cognitive impairment, Parkinson's disease, cancer, and obesity, and more preferably pediatric epilepsy, refractory epilepsy, glucose transporter 1 (GLUT1) deficiency, Alzheimer's disease, mild cognitive impairment, sarcopenia, and frailty. Examples of the prevention or amelioration of the obesity include actions that can be expected by increasing the blood acetoacetic acid concentration, for example, prevention or amelioration of obesity by suppressing weight gain, suppressing body fat accumulation, and promoting decomposition.

Furthermore, the ketone body production promoting composition of the present invention can be used in a method for increasing the blood ketone body (preferably acetoacetic acid) concentration of humans or non-human animals; and a method for treating, preventing, and ameliorating the above-mentioned diseases and/or symptoms. These methods include a step of administering an effective amount of the ketone body production promoting composition of the present invention to a subject (human or non-human animal, preferably a mammal), and more preferably include a step of administering to a subject with a disease or symptom in which an increase in blood ketone body concentration is effective.

The ketone body production promoting composition of the present invention can be administered to humans or non-human animals (preferably mammals) by either oral or parenteral routes. Note that, in the present invention, oral administration includes ingestion of food and drink compositions and feed compositions. The ketone body production promoting composition of the present invention may be, for example, a pharmaceutical composition, a quasi-drug composition, a food and drink composition, a feed composition, or the like, depending on the purpose, subject, method, dose, and the like for administering the composition.

The pharmaceutical composition and quasi-drug composition according to the present invention may be, for example, a preparation, and the form thereof is not particularly limited, and examples thereof include solids such as tablets, pills, granules, powders, powder preparations, and capsules; liquids such as general solutions, suspensions, emulsions, and syrups; jellies; injections and infusions; tube-administered agents and nasal-administered agents; and suppositories. The preparation can be produced according to a known method or a method similar thereto by, for example, blending the above-mentioned C6 or less fatty acid glyceride according to the present invention with one or more preparation auxiliary agents such as solvents, dispersants, emulsifiers, thickeners, gelling agents, surfactants, buffers, stabilizers, preservatives, excipients, binders, disintegrants, solubilizers, lubricants, colorants, flavor modifiers, sweeteners, coating agents, and perfumes.

In addition, as long as the effects of the present invention are not impaired, the pharmaceutical composition and quasi-drug composition may each contain an appropriate amount of one of additives such as water, lipids other than the C6 or less fatty acid glyceride according to the present invention, the above-mentioned sugars, the above-mentioned proteins, sugar alcohols, minerals (such as calcium, magnesium, sodium, potassium, iron, copper, and zinc), vitamins (such as vitamins A, Bl, B2, B6, B12, C, D, E, and K), peptides, amino acids, organic acids, and pH regulators alone, or the combination of two or more thereof.

The form of the food and drink composition according to the present invention is not particularly limited, and examples of the form include solids such as bars, liquids such as beverages and liquid foods, pastes, semi-liquids, gels (jellies), gel-like oils and/or fats (semi-solid oils and/or fats), and powders. In addition, the food and drink composition can also be used for nutritional management of oral and intestinal nutrition patients, the elderly, infants, and the like in the form of liquid foods, powdered liquid foods, nutritional pastes, oral and tube nutritional supplements, beverages, gelled foods, and the like.

Examples of the food and drink composition according to the present invention are not particularly limited, and include, for example, beverages (such as tea, carbonated beverages, cocoa, coffee, lactic acid bacteria beverages, soy milk beverages, fruit and vegetable juice beverages, soft drinks, nutritional beverages, and alcoholic beverages), processed foods (such as chocolate, gum, gummies, jellies, baked confectioneries (such as bread, cakes, cookies, and biscuits), and candy), dairy products (such as modified milk powder (milk powder), modified milk, milk beverage, fermented milk, yogurt, ice cream, cheese, cream, butter, margarine, and condensed milk), seasonings (such as sauces, soups, dressings, mayonnaise, mayonnaise-type seasonings, and creams), supplements, cooking oils, and functional edible oils and/or fats. Such food and drink compositions can be produced, for example, by a method of blending an existing food or drink with the C6 or less fatty acid glyceride according to the present invention, or a method of adding the C6 or less fatty acid glyceride according to the present invention in the process of producing the food or drink, and as the C6 or less fatty acid glyceride, the above preparation may be used as a raw material.

In addition, the food and drink composition according to the present invention may further contain various components that can be contained in the food and drink as long as the effects of the present invention are not impaired. Such components are not particularly limited, and may be contained, for example, in an appropriate amount of one of the preparation auxiliary agents and additives listed in the pharmaceutical compositions and quasi-drug compositions, dietary fibers (such as indigestible dextrin), fruits and vegetables and processed products thereof, animal and plant crude drug extracts, and naturally occurring macromolecules (such as collagen, hyaluronic acid, and chondroitin) alone, or in combination of two or more thereof.

In addition, examples of the food and drink composition according to the present invention include general food, health food, functional food, food with health claims (such as food for specified health uses, food with nutrient function claims, dietary supplement, and food with functional claims), food for special dietary uses (such as food for infants, food for pregnant women, and food for the sick), medical foods (foods prescribed under the supervision of doctors as defined by the US Food and Drug Administration (FDA) and the Orphan Drug Act), therapeutic food (which serves the purpose of treatment and is cooked based on the menu prepared by a dietitian or the like according to the dietary slip by the doctor), and dietary food, and the food and drink composition may be labeled with various actions and effects brought about by the C6 or less fatty acid glyceride according to the present invention in the product (for example, ketone body production promoting, blood ketone body concentration increasing, and alleviation, maintenance, improvement, and the like of diseases for which these are effective and symptoms related to the diseases).

Examples of the feed composition according to the present invention include those obtained by appropriately modifying the above-mentioned food and drink composition according to the purpose, subject, method, dose, and the like of giving the feed composition.

In addition, the dose of the ketone body production promoting composition of the present invention cannot be unequivocally determined because it is appropriately determined according to the individual case in consideration of the subject species, age, body weight, sex, difference in disease, degree of symptoms, and the like, but usually, for the amount of C6 or less fatty acid glyceride according to the present invention (in the case of a mixture of two or more kinds, the total amount thereof, the same applies hereinafter), the lower limit value per day for an adult can be, for example, 0.1 g, preferably 0.5 g, and more preferably 1 g. In addition, the upper limit value of the dose can be 100 g, preferably 80 g, and more preferably 60 g, and these lower limit values and upper limit values can be arbitrarily combined (for example, 0.1 to 100 g, preferably 0.5 to 80 g, and more preferably 1 to 60 g).

In addition, as the dose of the ketone body production promoting composition of the present invention, when the total amount of energy ingested by the subject is set to 100 kcal, the lower limit value of the calorie content of the ketone body production promoting composition can be, for example, 1 kcal, preferably 3 kcal, and more preferably 10 kcal. In addition, the upper limit value of the dose can be 100 kcal, preferably 90 kcal, and more preferably 85 kcal, and these lower limit values and upper limit values can be arbitrarily combined (for example, 1 to 100 kcal, preferably 3 to 90 kcal, and more preferably 10 to 85 kcal).

The ketone body production promoting composition of the present invention is preferably packaged (preferably enclosed) in a packaging container from the time of production to the time of administration. The packaging container is not particularly limited, and examples thereof include wrapping paper, packaging bags, soft bags, tubes, cheer packs, paper containers, cans, bottles, and capsules.

### EXAMPLES

Hereinafter, the present invention is described in more detail based on Examples, Reference Example, and Comparative Examples, but the present invention is not limited to the following Examples.

### Example 1

Butyric acid (having 4 carbon atoms (C4)) triglyceride (manufactured by Tokyo Chemical Industry Co., Ltd.) and water were mixed so that the mass ratio (butyric acid triglyceride: water) was 1: 2, and an emulsifier (soy lecithin) was added thereto so as to have a final concentration of 2% by mass and emulsified to prepare a test composition.

### Example 2

A test composition was prepared in the same manner as in Example 1 except that caproic acid (C6) triglyceride (manufactured by Tokyo Chemical Industry Co., Ltd.) was used instead of butyric acid triglyceride.

### Reference Example 1 (Ref.Ex.1)

A test composition was prepared in the same manner as in Example 1 except that caprylic acid (C8) triglyceride (manufactured by Tokyo Chemical Industry Co., Ltd.) was used instead of butyric acid triglyceride.

### Comparative Example 1 (Comp.Ex.1)

A test composition was prepared in the same manner as in Example 1 except that capric acid (C10) triglyceride (manufactured by Tokyo Chemical Industry Co., Ltd.) was used instead of butyric acid triglyceride.

### Comparative Example 2 (Comp.Ex.2)

A test composition was prepared in the same manner as in Example 1 except that lauric acid (C12) triglyceride (manufactured by Tokyo Chemical Industry Co., Ltd.) was used instead of butyric acid triglyceride.

### Comparative Example 3 (Comp.Ex.3)

A test composition was prepared in the same manner as in Example 1 except that palmitic acid (C14) triglyceride (manufactured by Tokyo Chemical Industry Co., Ltd.) was used instead of butyric acid triglyceride.

### Comparative Example 4 (Comp.Ex.4)

Water was used as it was as a test composition.

[Conformation Test for Ketone Body Production Promoting Effect]

Six-week-old male Wistar rats were given CLEA Rodent Diet CE-2 (CLEA Japan, Inc.) for one week for acclimatization, and then fasted overnight (16 hours or more and 20 hours or less). After the fasting, they were divided into seven groups of six in each group so that the average body weight was as equal as possible between the groups. After the grouping, each group was given a corresponding one of the test compositions prepared in Examples 1 and 2, Reference Example 1, and Comparative Examples 1 to 4. The dose was 3 g/kg body weight. From the tail veins of the rats, 100 μL of blood was collected before (0 hours) and 1, 2, 3, 4, 5, 6, 7, and 8 hours after administration of each test composition.

After separation of the plasma from the collected blood, Ketrex “Sanwa” (manufactured by Sanwa Kagaku Kenkyusho Co., Ltd.) was used to measure the plasma acetoacetic acid concentration and β-hydroxybutyric acid concentration, and the average measured values per group (of 6) of those measured values were defined as plasma acetoacetic acid concentration and plasma β-hydroxybutyric acid concentration, respectively. In addition, the sum of the plasma acetoacetic acid concentration and the plasma β-hydroxybutyric acid concentration was defined as the total plasma ketone body concentration. From the obtained results, a plasma concentration-time curve was obtained with the x-axis as the post administration elapsed time (unit: hr) and the y-axis as the plasma acetoacetic acid concentration, plasma β-hydroxybutyric acid concentration, or the total plasma ketone body concentration (unit: mmol/L), and the maximum plasma concentration of the curve between 0 and 8 hours was denoted by Cmax (unit: mmol/L), the integral value (area under the plasma concentration-time curve) was denoted by AUC₀₋₈ (unit: mmol·hr/L), and the integral value (area under the plasma concentration-time curve) between 0 and 4 hours of the curve was denoted by AUC₀₋₄ (unit: mmol·hr/L).

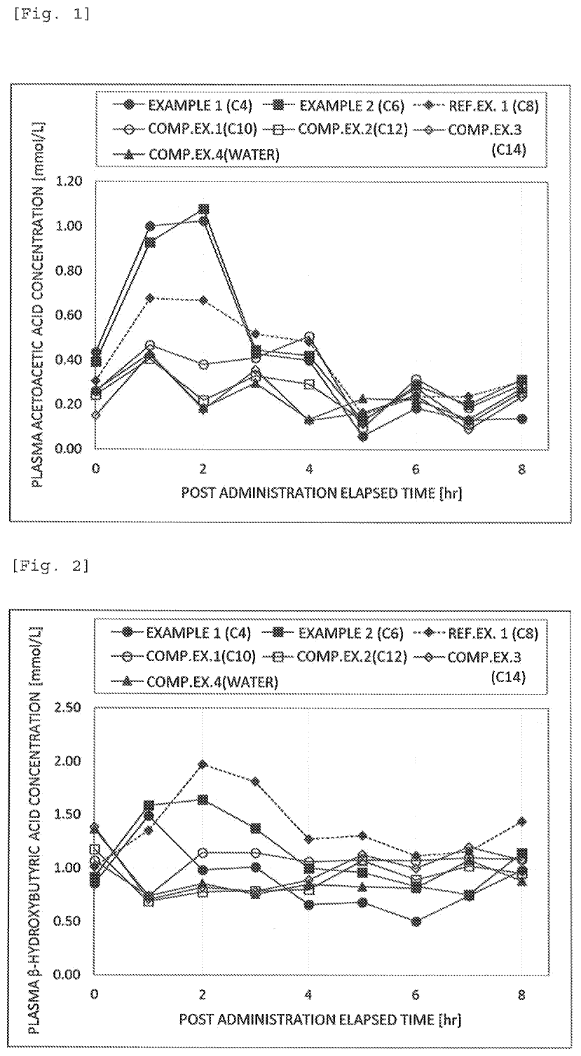

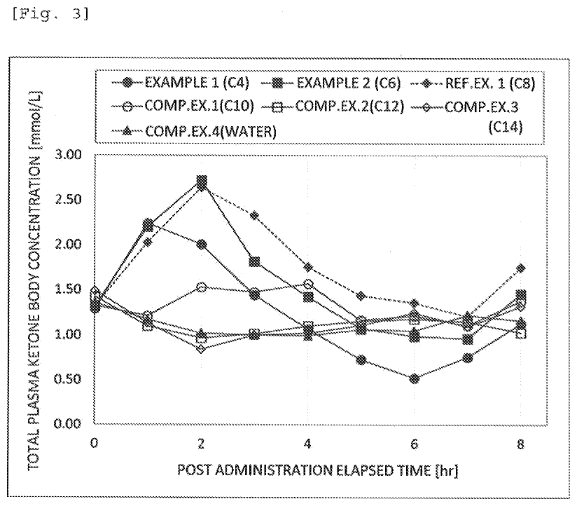

FIG. 1 shows a plasma acetoacetic acid concentration-post administration elapsed time curve obtained by administering the test compositions prepared in Examples 1 and 2, Reference Example 1, and Comparative Examples 1 to 4 to rats, FIG. 2 shows a plasma β-hydroxybutyric acid concentration-post administration elapsed time curve, and FIG. 3 shows a total plasma ketone body concentration-post administration elapsed time curve. In addition, Table 1 below shows Cmax, AUC₀₋₈, and AUC₀₋₄ in the plasma acetoacetic acid concentration-post administration elapsed time curve, and Table 2 below shows Cmax, AUC₀₋₈, and AUC₀₋₄ in the plasma β-hydroxybutyric acid concentration-post administration elapsed time curve. The following water group shows the group given the test composition prepared in Comparative Example 4.

TABLE 1

| | Plasma Acetoacetic Acid Concentration | | |
|---|---|---|---|
| | Cmax | AUC₀₋₈ | AUC₀₋₄ |
| | [mmol/L] | [mmol · hr/L] | [mmol · hr/L] |
| Example 1 (C4) | 1.1 ± 0.2* | 3.6 ± 0.6** | 2.9 ± 0.5* |
| Example 2 (C6) | 1.1 ± 0.3^{a} | 3.8 ± 0.7** | 2.8 ± 0.9* |
| Ref.Ex.1 (C8) | 0.7 ± 0.1^{a} | 3.4 ± 0.4** | 2.3 ± 0.3* |
| Comp.Ex.1 (C10) | 0.5 ± 0.1 | 2.6 ± 0.5^{a} | 1.6 ± 0.3 |
| Comp.Ex.2 (C12) | 0.4 ± 0.1 | 2.1 ± 0.3 | 1.2 ± 0.1 |
| Comp.Ex.3 (C14) | 0.4 ± 0.1 | 1.7 ± 0.3 | 1.0 ± 0.2 |
| Comp.Ex.4 | 0.4 ± 0.1 | 1.9 ± 0.3 | 1.1 ± 0.2 |
| (Water) | | | |

**p < 0.01; *p < 0.05; ^{a}p < 0.1, vs. water group
(Dunnett's test or Steel's test)

TABLE 2

| | Plasma β-Hydroxybutyric Acid | | |
|---|---|---|---|
| | Concentration | | |
| | Cmax | AUC₀₋₈ | AUC₀₋₄ |
| | [mmol/L] | [mmol · hr/L] | [mmol · hr/L] |
| Example 1 (C4) | 1.5 ± 0.3 | 7.0 ± 1.4 | 4.2 ± 0.4 |
| Example 2 (C6) | 1.7 ± 0.3 | 9.0 ± 1.9^{a} | 5.4 ± 1.1** |
| Ref.Ex.1 (C8) | 2.0 ± 0.4** | 11.2 ± 2.0** | 6.3 ± 1.2** |
| Comp.Ex.1 (C10) | 1.3 ± 0.2 | 8.5 ± 1.6 | 4.1 ± 0.8 |
| Comp.Ex.2 (C12) | 1.2 ± 0.2 | 7.0 ± 1.2 | 3.3 ± 0.6 |
| Comp.Ex.3 (C14) | 1.4 ± 0.2 | 7.9 ± 0.9 | 3.6 ± 0.5 |
| Comp.Ex.4 | 1.4 ± 0.3 | 7.0 ± 1.2 | 3.3 ± 0.7 |
| (Water) | | | |

**p < 0.01; ^{a}p < 0.1, vs. water group
(Dunnett's test or Steel's test)

As shown in FIGS. 1 to 3 and Tables 1 and 2, for the plasma β-hydroxybutyric acid concentration, no significant increase was observed after administration of other fatty acid glycerides (Comparative Examples 1 to 3) as compared with the water group (Comparative Example 4), but after administration of butyric acid triglyceride or caproic acid triglyceride (Examples 1 and 2), an increase was observed as in the case of administration of caprylic acid triglyceride (Reference Example 1). Also, for the plasma acetoacetic acid concentration as well, no significant increase was observed after administration of other fatty acid glycerides (Comparative Examples 1 to 3) as compared with the water group (Comparative Example 4), but after administration of butyric acid triglyceride or caproic acid triglyceride (Examples 1 and 2), a significant increase was observed, and the rate of increase was higher than that after administration of caprylic acid triglyceride (Reference Example 1). In addition, both the plasma β-hydroxybutyric acid concentration and the plasma acetoacetic acid concentration increased particularly during the 4 hours after administration. This confirms that the administration of a fatty acid glyceride containing a saturated fatty acid having 6 or less carbon atoms (C6 or less fatty acid glyceride) promotes the production of ketone bodies and increases the blood ketone body concentration. It has also been confirmed that the administration of the C6 or less fatty acid glyceride significantly increase the blood concentration of acetoacetic acid among the ketone bodies. It is presumed that this is because the production pattern of acetoacetic acid and the production pattern of β-hydroxybutyric acid are different.

### INDUSTRIAL APPLICABILITY

As described above, the present invention makes it possible to provide a ketone body production promoting composition capable of increasing the blood ketone body concentration, more specifically a pharmaceutical composition, a quasi-drug composition, a food and drink composition, and a feed composition, and more preferably a food and drink composition. In addition, according to the fatty acid glyceride (C6 or less fatty acid glyceride) containing a saturated fatty acid having 6 or less carbon atoms contained as an active ingredient in the ketone body production promoting composition of the present invention as a constituent fatty acid, gastrointestinal symptoms (such as diarrhea, vomiting, nausea, irritation, pain, fever, retching, bloating, and belch) are less likely to be caused by oral administration compared with fatty acid glycerides containing saturated fatty acids having 8 to 10 carbon atoms as constituent fatty acids, so that the gastrointestinal symptoms can be suppressed.

1. A ketone body production promoting composition comprising: as an active ingredient, a fatty acid glyceride which contains one to three molecules of fatty acids as constituent fatty acids and is a C6 or less fatty acid glyceride in which at least one molecule of the constituent fatty acids is a saturated fatty acid having 6 or less carbon atoms.

2. The ketone body production promoting composition according to claim 1, wherein the C6 or less fatty acid glyceride is a fatty acid triglyceride containing three molecules of fatty acids as constituent fatty acids.

3. The ketone body production promoting composition according to claim 1, wherein the amount of the saturated fatty acid having 6 or less carbon atoms as a constituent fatty acid of the C6 or less fatty acid glyceride is 5% by mass or more when a total amount of the constituent fatty acids of the fatty acid glyceride contained in the ketone body production promoting composition is set to 100% by mass.

4. The ketone body production promoting composition according to claim 1, wherein the saturated fatty acid having 6 or less carbon atoms has 4 to 6 carbon atoms in the C6 or less fatty acid glyceride.

5-7. (canceled)

8. The ketone body production promoting composition according to claim 1, which is a composition selected from the group consisting of pharmaceutical compositions, quasi-drug compositions, food and drink compositions, and feed compositions.

9. A method for increasing a blood ketone body concentration, comprising: administering the ketone body production promoting composition according to claim 1 to a human or non-human animal.

10. (canceled)

11. The method for increasing a blood ketone body concentration according to claim 9, wherein the method is a method for preventing or ameliorating a disease or symptom in which an increase in blood ketone body concentration is effective.

12-13. (canceled)

14. The method for increasing a blood ketone body concentration according to claim 9, wherein the C6 or less fatty acid glyceride is a fatty acid triglyceride containing three molecules of fatty acids as constituent fatty acids.

15. The method for increasing a blood ketone body concentration according to claim 9, wherein the amount of the saturated fatty acid having 6 or less carbon atoms as a constituent fatty acid of the C6 or less fatty acid glyceride is 5% by mass or more when a total amount of the constituent fatty acids of the fatty acid glyceride contained in the ketone body production promoting composition is set to 100% by mass.

16. The method for increasing a blood ketone body concentration according to claim 9, wherein the saturated fatty acid having 6 or less carbon atoms has 4 to 6 carbon atoms in the C6 or less fatty acid glyceride.

17. The method for increasing a blood ketone body concentration according to claim 9, wherein the ketone body is acetoacetic acid, and the method comprising promoting production of acetoacetic acid.

18. The method for increasing a blood ketone body concentration according to claim 14, wherein the amount of the saturated fatty acid having 6 or less carbon atoms as a constituent fatty acid of the C6 or less fatty acid glyceride is 5% by mass or more when a total amount of the constituent fatty acids of the fatty acid glyceride contained in the ketone body production promoting composition is set to 100% by mass.

19. The method for increasing a blood ketone body concentration according to claim 14, wherein the saturated fatty acid having 6 or less carbon atoms has 4 to 6 carbon atoms in the C6 or less fatty acid glyceride.

20. The method for increasing a blood ketone body concentration according to claim 14, wherein the ketone body is acetoacetic acid, and the method comprising promoting production of acetoacetic acid.

21. The method for increasing a blood ketone body concentration according to claim 15, wherein the saturated fatty acid having 6 or less carbon atoms has 4 to 6 carbon atoms in the C6 or less fatty acid glyceride.

22. The method for increasing a blood ketone body concentration according to claim 15, wherein the ketone body is acetoacetic acid, and the method comprising promoting production of acetoacetic acid.

23. The method for increasing a blood ketone body concentration according to claim 16, wherein the ketone body is acetoacetic acid, and the method comprising promoting production of acetoacetic acid.

24. The method for increasing a blood ketone body concentration according to claim 18, wherein the saturated fatty acid having 6 or less carbon atoms has 4 to 6 carbon atoms in the C6 or less fatty acid glyceride.

25. The method for increasing a blood ketone body concentration according to claim 18, wherein the ketone body is acetoacetic acid, and the method comprising promoting production of acetoacetic acid.

26. The method for increasing a blood ketone body concentration according to claim 24, wherein the ketone body is acetoacetic acid, and the method comprising promoting production of acetoacetic acid.
